(19) United States
(12) Patent Application Publication
(10) Pub. No.: US 2014/0094927 A1
(43) Pub. Date: Apr. 3, 2014

(54) Joint Prosthesis with Polymeric Articulation Interface

(71) Applicant: Steven H. Weeden, Fort Worth, TX (US)

(72) Inventor: Steven H. Weeden, Fort Worth, TX (US)

(21) Appl. No.: 14/035,387

(22) Filed: Sep. 24, 2013

Related U.S. Application Data
(60) Provisional application No. 61/708,421, filed on Oct. 1, 2012.

(51) Int. Cl. A61F 2/34; A61F 2/36

(52) U.S. Cl. CPC A61F 2/34; A61F 2/3609
USPC 623/22.21; 623/22.4; 623/23.11

(57) ABSTRACT

A hip arthroplasty prosthesis for treating a patient condition includes a stem portion configured to penetrate into the bone of a femur of the patient, and includes a head portion formed of a polymeric material and being fixedly attached to the stem portion. The head portion may include a spherical portion with a diameter sized to match the diameter of an acetabulum of an adult patient in a manner permitting the acetabulum to interface with and articulate relative the head portion over a distributed portion of the head portion.

### PRIORITY

This patent application claims priority to and the benefit of the filing date of U.S. Provisional Patent Application 61/708,421, titled “Joint Prosthesis with Polymeric Articulation Interface,” filed Oct. 1, 2012, which is incorporated herein by reference.

### FIELD OF THE INVENTION

This disclosure is directed to prostheses for use in partial or full total joint replacement (arthroplasty).

### BACKGROUND

Various prostheses have been proposed to provide relief and mobility to a patient. For example, full or total and partial hip replacement prostheses are successfully used to treat arthritis pain and repair joint damage, such as damage that may present after osteoarthritis or as a result of a hip fracture.

A full hip replacement (total hip arthroplasty) typically includes replacing the acetabulum with a metal cup, and replacing the proximal femur and the femoral head with a pivoting ball. The ball then articulates in the acetabular cup creating a ball and socket joint. A partial hip replacement typically includes replacing only the femoral head. The artificial femoral head fits within the native acetabulum to provide an articulating joint.

Hospitals and clinics that treat these conditions and patients requiring joint replacement frequently store expensive inventory in order to have required implants on hand if and when the need arises. Typically, each type of treatment requires specific implants, preparatory tools, and supplies. Large inventories result in heavy financial outlays by hospitals and clinics who may not receive reimbursement until these prostheses are used.

The present disclosure is directed to devices, systems, and methods that address one or more deficiencies in the prior art.

### SUMMARY

In one exemplary aspect, the present disclosure is directed to a hip arthroplasty prosthesis for treating a patient condition (arthroplasty). The prosthesis includes a stem portion configured to penetrate into the bone of a femur of the patient, and includes a head portion formed of a polymeric material and being fixedly attached to the proximal stem portion. The head portion may include a spherical portion with a curvature measured as a radius or diameter sized to match the curvature measured as a radius or diameter of an acetabulum of an adult patient in a manner permitting the acetabulum to interface with and articulate relative the head portion over a distributed portion of the head portion.

In some aspects, a distributed portion of the head portion means the diameter of the head portion and the diameter of the acetabulum differ by about 15% or less. The diameter of the head portion and the diameter of the acetabulum differ by about 10% or less. In one aspect, the polymeric material of the head portion is ultra-high high molecular weight polyethylene.

In some aspects, an acetabular cup includes an outer surface configured to interface with the acetabulum and an inner surface configured to interface with the head portion over a distributed portion of the head portion.

In some aspects, the head portion has a diameter within a range of about 28-48 mm. In most aspects, the stem portion is configured to extend into the intramedullary canal of the femur.

In some aspects, the head portion comprises a tapering portion configured to minimize tissue damage when the prosthesis articulates relative to an acetabulum. In some aspects, the tapered portion is an annular taper disposed adjacent the neck.

In another exemplary aspect, the present disclosure is directed to a surgical technique for implanting a hip arthroplasty prosthesis to treat a patient condition. The method may include anchoring a prosthetic head portion to a femoral portion of a patient so that the head portion is fixed. The head portion may be formed of a polymeric material and may be fixedly attached to the femoral portion. The head portion may have a spherical portion with a diameter sized to match the diameter of an acetabulum of an adult patient in a manner permitting the acetabulum to interface with and articulate relative the head portion over a distributed portion of the head portion. The method may include inserting the head portion into the acetabulum so that the polymeric head portion interfaces directly with the acetabulum in an articulating relationship. In one aspect, the polymeric material of the head portion is ultra-high molecular weight polyethylene.

In another exemplary aspect, the present disclosure is directed to a surgical technique for implanting a hip arthroplasty prosthesis to treat a patient condition. The method may include anchoring a prosthetic head portion to a femoral portion of a patient so that the head portion is immoveable relative to the femoral portion. The head portion may be formed of a polymeric material, and may have a spherical portion with a diameter sized to match the diameter of an acetabulum of an adult patient in a manner permitting the acetabulum to interface with and articulate relative the head portion over a distributed portion of the head portion. The method may also include forming the acetabulum to receive an acetabular cup comprising an outer surface configured to interface with the acetabulum and an inner surface configured to interface with the head portion over a distributed portion of the head portion, and may include inserting the head portion into the acetabular cup so that the polymeric head portion interfaces directly with the acetabular cup in an articulating relationship.

### BRIEF DESCRIPTION OF THE DRAWINGS

Aspects of the present disclosure are best understood from the following detailed description when read with the accompanying figures.

FIG. 1 is an illustration of a partial hip arthroplasty prosthesis in accordance with one exemplary aspect of the present disclosure.

FIG. 2 is an illustration of a full or total hip arthroplasty prosthesis in accordance with one exemplary aspect of the present disclosure.

### DETAILED DESCRIPTION

The following disclosure provides many different embodiments, or examples, for implementing different features of various embodiments. Specific examples of components and arrangements are described below to simplify the present disclosure. These are, of course, merely examples and are not intended to be limiting. In addition, the present disclosure may repeat reference numerals and/or letters in the various examples. This repetition is for the purpose of simplicity and clarity and does not in itself dictate a relationship between the various embodiments and/or configurations discussed.

The devices, systems, and methods described herein include features that increase the overall simplicity and functionality of joint restructuring or replacement implants that may be used in full hip replacement or a partial hip replacement in both original and revision surgeries. The devices, systems, and methods described herein employ a unique prosthesis arrangement using a solid highly-crosslinked polyethylene polymer articulation ball that is modular in that it may be used as the articulation ball in a partial hip replacement surgery and may also be used in a full hip replacement surgery with an acetabular cup. In some exemplary aspects, a single articulation ball sized and arranged to be used in both surgeries may enable hospitals and clinics to decrease overall inventory.

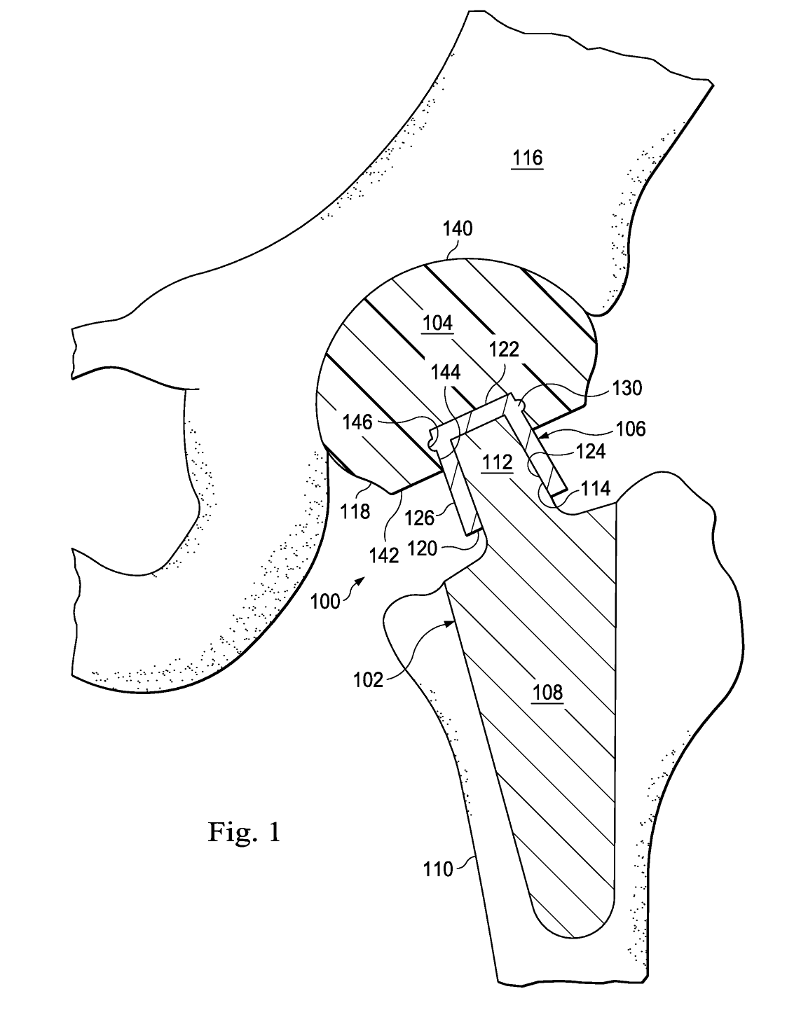

FIG. 1 shows a first embodiment of a hip prosthesis 100 usable in a partial hip replacement. The hip prosthesis includes a stem component 102, a ball or head portion 104, and a connector 106. In this example, the stem 102 includes an anchor portion 108 sized to fit at least partially within the intramedullary canal of a femur 110 and includes a neck portion 112 extending from the anchor portion 108. In the example of FIG. 1, the neck portion 112 is received within a receiving bore (morse-taper) 114 on the connector 106. The stem 102 may be formed of any suitable material, and is preferably formed of a biocompatible materials, such as cobalt chrome, oxidized zirconium, or titanium, for example.

In this embodiment, the connector 106 is rigidly fixed to both the stem component 102 and the head portion 104. The connector 106 is shaped as a cap or thimble and includes an open end 120 and a closed end 122.

The connector 106 is configured to receive the neck portion 112 within its open end 120. The connector 106 includes an inner surface 124 forming the receiving bore 114 and an outer surface 126. In the exemplary embodiment shown, the inner and outer surfaces 124, 126 are each formed with a taper, such as a morse taper, a 12-14 taper, or a C-taper. For example, the inner surface 124 may have a diameter in a range of about 10-16 mm adjacent the open end, and may have a diameter in range of about 8-14 mm near the distal end. Of course, other sizes are contemplated. In some embodiments, the neck portion 112 of the stem 102 tapers or has a frusto-conical shape with a diameter matching the diameter of the connector 106. The outer surface 126 of the connector 106 may extend from the open end 120 tapering toward the closed end 122.

In some embodiments, the outer diameter of the connector 106 includes a locking element 130 shown as a protruding lip on the outer surface 126. The locking element 130 extends radially outwardly on the outer surface 126 of the connector 106 and is configured to connect the connector 106 to the head portion 104 in a manner described further below. In the embodiment shown, the locking mechanism 130 is disposed adjacent the closed end 122. In other embodiments, the locking mechanism 130 is more centrally disposed along the outer surface 126 of the connector 106.

A biocompatible cement, adhesive, or other attachment may be used to secure the head portion 104 to the connector 106 and to secure the connector 106 to the stem component 102. In some embodiments, the stem component 102 and the connector 106 are formed together as a monolithic component (morse taper fit).

The head portion 104 includes a distal portion 140 and a proximal portion 142, with the distal portion 140 forming an outer articulation surface and proximal portion 142 including a recess 144 formed therein. As indicated above, the distal portion 140 may have a radius in the range of about 14-24 mm. The recess 144 is configured to receive the closed end 122 of the connector 106 in the manner shown in FIG. 1. In the exemplary embodiment shown, the recess 144 includes a radially extending locking feature, formed of a radial groove 146 in the recess 144. The radial groove 146 is configured to receive the locking element 130 on the connector 106 and is configured to secure the head portion 104 to the connector 106. As can be seen, when the locking mechanism 130 on the connector 106 is disposed within the radial groove 146 in the recess of the head portion 104, the head portion 104 is secured to the connector 106 via mechanical interference. While FIG. 1 shows the locking mechanism as the protruding lip on the connector 106 and the radial groove on the head portion 104, other embodiments include a protruding lip on the head portion 104 and the radial groove on the connector 106. Yet other locking systems are contemplated. In addition, in some embodiments, the connector 106 and the neck portion 112 also include a locking system, such as a protruding lip and groove that secure the connector to the neck portion. In embodiments where the connector 106 and the neck portion 112 are integrally formed, the resulting outer surface may cooperate directly with the head portion 104 as described above to secure the head portion 104 to the stem component 102.

The head portion 104 is formed of a solid polymeric (highly crosslinked polyethylene) material in the exemplary embodiment shown in FIG. 1. Preferably, the polymeric material has a modulus that is closer to that of bone than that of a ceramic, an alumina carbide, or a metal such as titanium. In some embodiments, the polymeric material has a hardness that is within the range of about 4-6 on the Mohs Hardness Scale, and in some embodiments, is close to the hardness of a natural bone material that may be found on a natural femoral head. In some embodiments, the head portion 104 has an articulating portion that is at least partially spherical shaped. It may interface with the acetabulum 116 of the patient. Some exemplary embodiments include a tapered rim 118 to decrease the possibility of soft tissue or muscle irritation or impingement.

In a preferred embodiment, the head portion 104 is formed of ultra-high molecular weight polyethylene (UHMWP). UHMWP has a modulus of elasticity less than that of a metal as described above and provides a smooth articulation interface with the bone material of the acetabulum 116. Preferably the head portion 104 has a diameter sized similar to or slightly less than that of the acetabulum 116 so that articulation rides on a distributed portion of the head portion and the acetabulum. As used herein, a distributed portion means the head portion 104 and the acetabulum have a 15% or less difference in diameters. In some embodiments, the head portion 104 may have a diameter sized to match the diameter of an acetabulum of an adult patient. Thus, the head portion may have a diameter in the range of, for example, about 28-48 mm. In some embodiments, the interfacing surface of the head portion 104 is formed with a radius in the range of about 14-24 mm. In some embodiments, the head portion 104 and the acetabulum have a 10% or less difference in diameters, and in others, the head portion 104 and the acetabulum have a 5% or less difference in diameters. Because the articulation interface includes a prosthetic material that is less rigid than metal, the interface may be longer lasting and result in less trauma (to the host acetabular cartilage) than in prior systems where the articulation interface is metal on bone. For example, because of the rigidity of the metal/bone interface in a prior art device, the bone alone is required to deform or is damaged by the stiffness and shape of the metal ball. However, one advantage achieved by the proposed head portion formed of a polymeric material, such as UHMWP is that the head portion may be slightly resilient and may slightly deform under compressive loads. Accordingly, the bone and the head portion 104 together may deform as necessary to accommodate loading. This reduced trauma at the bone/implant interface may provide a more comfortable interface for the patient over a longer period of time, and may result in decreased morbidity and decreased rate of future revision surgery.

In other embodiments, the head portion 104 is formed of additional or alternative biocompatible materials including, for example, polyetheretherketone resins (“PEEK”), carbon reinforced PEEK, ultra high molecular weight polyethylenes, polyethylenes, polyanhydrid, and alpha polyesters. Other materials are contemplated.

In addition, as can be seen in FIG. 1, the head portion 104 is formed of substantially solid piece or of a mono-block of the polymeric material. This permits the head portion 104 to strain further, permitting the head portion 104 to more easily accommodate imperfections in the bone material forming the acetabulum 116. Along the proximal portion of the head portion 104 is the tapered rim 118 that deviates from the spherical outer surface of the head portion 104. The tapered rim 118 is a relief providing spacing from the bone and inhibits or reduces the change of tissue irritation that may otherwise occur as the head portion 104 articulates relative to the bone at the extreme limits of rotation. In some embodiments, the tapered rim 118 is formed of a conical shape, while in other embodiments, the tapered rim 118 has a curve at an angle different than the spherical outer surface so that the tapered rim 118 provides spacing from the bone. In some embodiments, the tapered rim 118 extends from the spherical outer surface to the proximal portion 142, which may be a relatively flat or planar surface forming the non-articulating side of the head portion. In some examples, the polymeric head portion 104 is fitted with radiopaque markers that enable the head portion to appear on radiographs, such as x-rays.

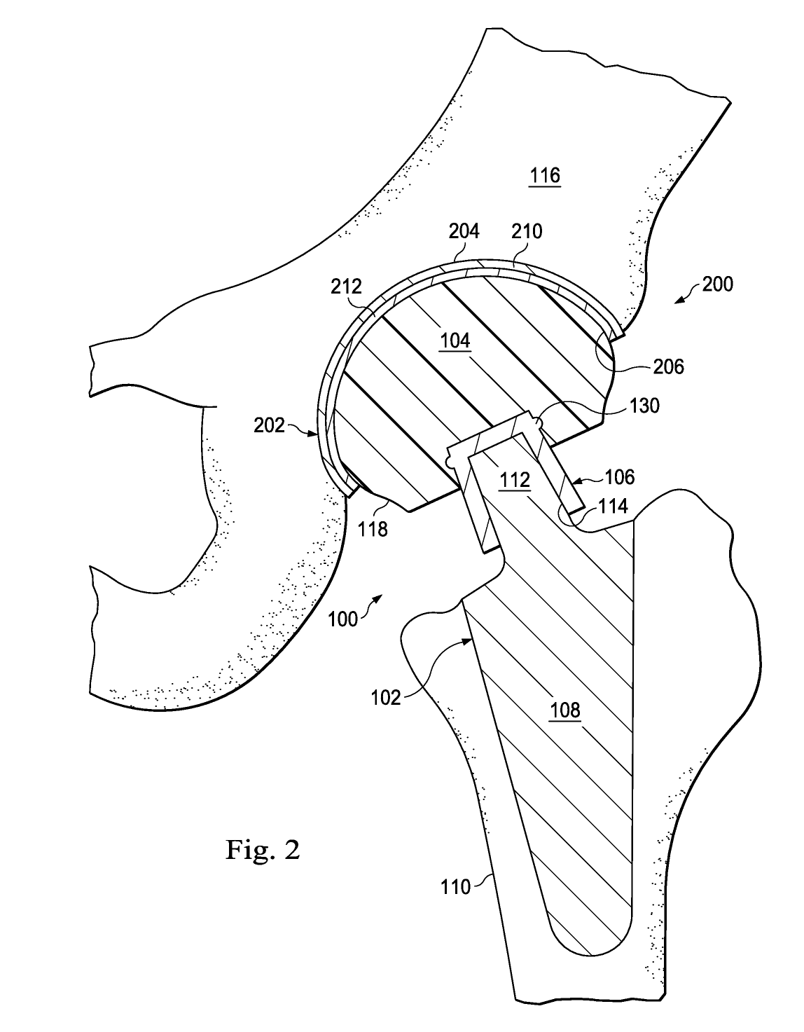

FIG. 2 shows the partial hip prosthesis 100 in combination with a prosthetic acetabular cup, together forming a full hip replacement prosthesis, referenced herein by the numeral 200. The hip prosthesis 200 includes an acetabular cup 202 that is formed of a metal material, such as titanium, Oximium, alloys, cobalt-chrome, or other biocompatible metals and alloys. In some embodiments, the acetabular cup 202 is formed of a ceramic material. The acetabular cup 202 includes an outer facing surface 204 and an inner facing or concave surface 206. The outer facing surface 204 is configured to interface with the host acetabular bone. It may be secured using methods known in the art, including using fixation elements, such as screws, anchors, cleats, other surface deformations including, but not limited to, for example, a porous coating. In some examples the acetabular cup 202 may be held in place using cements, biocompatible adhesives, or other fixation methods.

The inner facing surface 206 is typically smooth and provides a smooth articulating interface with the head portion 104. Because of its material, the cup 202 is harder and less rigid than the polymeric head portion 104. In this embodiment, the cup 204 includes a shell 210 and a liner 212, with the inner facing surface 206 formed as a surface of the liner 212 and the outer facing surface formed by the shell 210. Both the shell 210 and the liner 212 may be formed of a metal material, and the liner 212 is fixed relative to the shell 210 to prevent articulating between the liner 212 and shell 210. Although a single liner 212 is shown, other embodiments include two liners, and yet other embodiments include more than two liners. In some embodiments, the acetabular cup 202 includes only a shell without a liner.

One advantage of the system disclosed herein is that there is no metal-to-metal articulating surface. Therefore, the overall implant is less rigid than metal on metal bearings, avoids the binding that may occur over time in some metal to metal interfaces, avoids the potential for excessive metal debris and subsequent host physiologic reaction, and provides some cushioning. Numerous articles report the negative results and failures of metal on metal articulation in total hip arthroplasty. The system disclosed herein provides the large size of articulation desired without the potential risk of metal debris and failure or host physiologic response.

In the example shown the cup 202 has a thickness within a range of about 4-8 mm. Of course, other sizes are contemplated. The head portion 104 has a diameter in the range of about 28-48 mm (sizes may vary yet will typically be 6-10 mm less than the OD (outer diameter) of the acetabular shell). Of course, other sizes, both larger and smaller are contemplated. Because of this, the head portion 104 is substantially the same size as the head portion 104 used in the partial hip replacement. In one aspect, the same head portion 104 may be used in either the partial hip replacement or the full hip replacement, as is discussed further below.

The embodiments disclosed herein include a single articulation surface. As such, the embodiments may provide advantages over systems having dual articulation surfaces. For example, all the articulation occurs at the greatest available diameter. Accordingly, the articulation is smoother and more stable than articulation that might occur using dual diameter designs or using articulations with smaller diameters. Furthermore, since all the articulation occurs at the diameter that substantially matches the original femoral head, the articulation is more stable and more completely mimics that of a natural hip joint.

In some aspects, the present disclosure is directed to a system that reduces necessary inventory for hospitals and clinics because the same partial hip replacement prosthesis may be used in a partial hip replacement surgery and may be used in a full hip replacement surgery. The only difference is that the full hip replacement surgery also uses the acetabular cup 202. Because both use the same partial hip replacement prosthesis, a hospital or clinic that maintains the full hip replacement prosthesis also then has the partial hip replacement prosthesis. This improvement may reduce inventory costs and may even conserve storage space at the hospitals and clinics.

In use, a surgeon may introduce the prosthesis to the patient to treat a medical condition, such as hip arthritis, a fractured hip or pelvis, or other condition. The stem portion 102 of the prosthesis 100 is implanted into the femur 110 using methods known in the discipline. Either before or after the stem is implanted, the head portion 104 is fixedly and immovably attached to stem portion 102. In the exemplary embodiment shown, this may be done by securing the stem 102 and the head portion 104 to the connector 106 using methods known in the art. Accordingly, the head portion 104 extends from the upper end or trochanter end of the femur 110 toward the acetabulum.

If the surgery is a partial hip replacement, the head is introduced to the acetabulum so that the polymeric head abuts against the concave bone structure of the acetabulum. As described above, the polymeric head is sized to fit within the acetabulum so that articulation occurs at the bone-polymer interface, and the polymeric head portion 104 is sized to match that of an adult human patient so that the interface is a distributed relationship. Since the articulation with the polymer/bone interface occurs only at the large diameter head portion 104, the articulation is stable and mimics that of a natural hip joint.

If the surgery is a full hip replacement, the acetabulum is shaped to receive the acetabular cup 202. The acetabular cup 202 is then introduced and secured in place using mechanical fixation, a cement or adhesive, ingrowth, or other methods. The polymeric head 104 is the introduced to the acetabulur cup 202 so that the polymeric head abuts against the inner surface 206 of the cup 202. As described above, the polymeric head portion 104 is sized to fit within the cup 202 so that articulation occurs at the metal-polymer interface, and the polymeric head portion is sized to match that of an adult human patient so that the interface is a distributed relationship. Since the articulation with the polymer/metal interface occurs only at the large diameter head portion, the articulation is stable and mimics that of a natural hip joint.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims. It has obvious implications in major complex revision and other reconstructive hip arthroplasty procedures.

I claim:

1. A hip arthroplasty prosthesis for treating a patient condition, comprising:
a stem portion configured to penetrate into the bone of a femur of the patient;
a head portion formed of a polymeric material and being fixedly attached to the stem portion, the head portion having a spherical portion with a diameter sized to match the diameter of an acetabulum of an adult patient in a manner permitting the acetabulum to interface with and articulate relative the head portion over a distributed portion of the head portion.

2. The hip arthroplasty prosthesis of claim 1, wherein a distributed portion of the head portion means the diameter of the head portion and the diameter of the acetabulum differ by about 15% or less.

3. The hip arthroplasty prosthesis of claim 2, wherein the diameter of the head portion and the diameter of the acetabulum differ by about 10% or less.

4. The hip arthroplasty prosthesis of claim 1, wherein the polymeric material of the head portion is ultra-high molecular weight polyethylene.

5. The hip arthroplasty prosthesis of claim 1, comprising:
an acetabular cup comprising an outer surface configured to interface with the acetabulum and an inner surface configured to interface with the head portion over a distributed portion of the head portion.

6. The hip arthroplasty prosthesis of claim 5, wherein the head portion has a diameter within a range of about 28-48 mm.

7. The hip arthroplasty prosthesis of claim 5, wherein the stem portion extends into the intramedullary canal of the femur.

8. The hip arthroplasty prosthesis of claim 1, wherein the head portion comprises a tapering portion configured to minimize tissue damage when the prosthesis articulates relative to an acetabulum.

9. The hip arthroplasty prosthesis of claim 8, further comprising a neck portion, the head portion being disposed on the neck portion, the tapered portion being an annular taper disposed adjacent the neck.

10. A surgical technique for implanting hip arthroplasty prosthesis to treat a patient condition, comprising:
anchoring a prosthetic head portion to a femoral portion of a patient so that the head portion is fixed the head portion being formed of a polymeric material and being fixedly attached to the femoral portion, the head portion having a spherical portion with a diameter sized to match the diameter of an acetabulum of an adult patient in a manner permitting the acetabulum to interface with and articulate relative the head portion over a distributed portion of the head portion; and
inserting the head portion into the acetabulum so that the polymeric head portion interfaces directly with the acetabulum in an articulating relationship.

11. The method of claim 10, wherein the polymeric material of the head portion is ultra-high high molecular weight polyethylene.

12. The method of claim 10, wherein a distributed portion of the head portion means the diameter of the head portion and the diameter of the acetabulum differ by about 15% or less.

13. The method of claim 12, wherein the diameter of the head portion and the diameter of the acetabulum differ by about 10% or less.

14. The method of claim 10, wherein the head portion has a diameter within a range of about 28-48 mm.

15. The method of claim 10, wherein the head portion comprises a tapering portion configured to minimize tissue damage when the prosthesis articulates relative to an acetabulum.

16. The method of claim 15, further comprising a neck portion, the head portion being disposed on the neck portion, the tapered portion being an annular taper disposed adjacent the neck.

17. A surgical technique for implanting a hip arthroplasty prosthesis to treat a patient condition, comprising:
anchoring a prosthetic head portion to a femoral portion of a patient so that the head portion is immoveable relative to the femoral portion, the head portion being formed of a polymeric material, the head portion having a spherical portion with a diameter sized to match the diameter of an acetabulum of an adult patient in a manner permitting the acetabulum to interface with and articulate relative the head portion over a distributed portion of the head portion;
forming the acetabulum to receive an acetabular cup comprising an outer surface configured to interface with the acetabulum and an inner surface configured to interface with the head portion over a distributed portion of the head portion; and
inserting the head portion into the acetabular cup so that the polymeric head portion interfaces directly with the acetabular cup in an articulating relationship.

18. The method of claim 17, wherein the polymeric material of the head portion is ultra-high high molecular weight polyethylene.

19. The method of claim 17, wherein the acetabular cup is metal.

20. The method of claim 17, wherein a distributed portion of the head portion means the diameter of the head portion and the diameter of the acetabulum differ by about 15% or less.

21. The method of claim 20, wherein the diameter of the head portion and the diameter of the acetabulum differ by about 10% or less.
